(19) United States
(12) Patent Application Publication
(10) Pub. No.: US 2016/0158383 A1
(43) Pub. Date: Jun. 9, 2016

(54) METHOD AND DEVICE FOR MANUFACTURING POLYMER PARTICLES CONTAINING A THERAPEUTIC MATERIAL

(71) Applicant: THE UNIVERSITY OF BRITISH COLUMBIA, Vancouver (CA)

(72) Inventors: Euan Ramsay, Vancouver (CA); Robert James Taylor, Vancouver (CA); Andre Wild, Vancouver (CA); Timothy Leaver, Delta (CA); Colin Walsh, Belmont, CA (US)

(73) Assignee: The University of British Columbia, Vancouver, BC (CA)

(21) Appl. No.: 14/907,507

(22) PCT Filed: Jul. 25, 2014
(86) PCT No.: PCT/US2014/048165

§ 371 Date: Jan. 25, 2016

Related U.S. Application Data
(60) Provisional application No. 61/858,973, filed on Jul. 26, 2013.

(51) Int. Cl. A61K 47/48; A61K 31/337

(52) U.S. Cl. CPC A61K 47/48923; A61K 31/337; A61K 47/48907

(57) ABSTRACT

Methods and devices for manufacturing polymer particles containing a therapeutic material and polymer conjugate particles comprising (a) introducing a first stream comprising a first solvent into a channel, wherein the channel has a first region adapted for flowing one or more streams introduced into the channel and a second region for mixing the contents of the one or more streams; (b) introducing a second stream comprising polymer conjugate in a second solvent into the channel to provide first and second streams flowing in the channel; (c) flowing the one or more first streams and the one or more second streams from the first region of the channel into the second region of the channel; (d) mixing of the contents of the one or more first streams and the one or more second streams flowing in the second region of the channel to provide a third stream comprising polymer conjugate nanoparticles.

### CROSS-REFERENCE TO RELATED APPLICATION

This application claims the benefit of U.S. patent application Ser. No. 61/858,973, filed Jul. 26, 2013, expressly incorporated herein by reference in its entirety.

### FIELD OF THE INVENTION

The present invention provides methods and devices for manufacturing polymer particles containing a therapeutic material.

### BACKGROUND OF THE INVENTION

A major challenge for many active pharmaceutical ingredients (therapeutic materials) is the inability to deliver adequate concentration to target cells to elicit a biological affect. Certain therapeutic materials, including many chemotherapeutic therapeutic materials, are toxic and cannot be administered systemically at doses that are required to have an affect on a disease, while others, including many biologics like oligonucleotide therapeutic materials, are unable to cross cell membranes to access their site of action. Polymer nanomaterials are a promising solution for encapsulating therapeutic materials and transporting therapeutic materials to diseased cells and tissues. Polymer nanoparticles can increase a therapeutic materials therapeutic index by reducing toxicity through shielding the therapeutic material from healthy tissues, increasing the therapeutic material effectiveness through targeting diseased tissue, and by enabling the active delivery of therapeutic materials to their site of action.

Polymer nanoparticles have been developed using a wide range of materials including synthetic homopolymers such as polyethylene glycols, polylactides, polyglycolides, polyacrylates, polymethacrylates, poly(ε-caprolactone)s, polyorthoesters, polyanhydrides, polylysines, polyethyleneimines; synthetic copolymers such as poly(lactide-co-glycolide)s, poly(lactide)-poly(ethylene glycol)s, poly(lactide-co-glycolide)-poly(ethylene glycol)s, poly(ε-caprolactone)-poly(ethylene glycol)s; and natural polymers such as celluloses, chitins, and alginates.

A variety of classes of therapeutic materials have been entrapped and/or associated with polymer nanoparticles for the purposes of improving therapeutic or diagnostic performance and disease outcomes including low molecular weight organic compounds, nucleic acids, proteins, peptides, inorganic elements and radioactive materials.

A variety of methods have been developed to manufacture polymer nanoparticles. These methods include self-assembly, nanoprecipitation, and homogenization.

Despite the availability of methods of manufacture for polymer nanoparticle systems, a need exist for improved methods and devices or preparing polymer nanoparticles containing therapeutic materials. The present invention seeks to fulfill this need and provides further related advantages.

### SUMMARY OF THE INVENTION

In one aspect, the invention provides methods for making polymer conjugate nanoparticles.

In one embodiment, the method includes:

(a) introducing a first stream (e.g., one or more first streams) comprising a first solvent into a channel, wherein the channel has a first region adapted for flowing one or more streams introduced into the channel and a second region for mixing the contents of the one or more streams;

(b) introducing a second stream (e.g., one or more second streams) comprising polymer conjugate in a second solvent into the channel to provide first and second streams flowing in the channel;

(c) flowing the one or more first streams and the one or more second streams from the first region of the channel into the second region of the channel; and

(d) mixing of the contents of the one or more first streams and the one or more second streams flowing in the second region of the channel to provide a third stream comprising polymer conjugate nanoparticles.

Polymer conjugates are polymers to which one or more molecular species (e.g., therapeutic agent) are covalently, or non-covalently, coupled (i.e., molecular species is conjugated to the polymer). Suitable polymers include natural polymers, synthetic polymers, semi-synthetic polymers, derivatives thereof, combinations thereof, and copolymers thereof. Representative polymers include polyethylene glycols, polylactides, polyglycolides, poly(lactide-co-glycolide)s, polyacrylates, polymethacrylates, poly(ε-caprolactone)s, polyorthoesters, polyanhydrides, polylysines, polyethyleneimines, celluloses, chitins, alginates, carboxymethylcelluloses, acetylated carboxymethylcelluloses, chitosans, and gelatins, derivatives thereof, combinations thereof, and copolymers thereof. Suitable molecular species include small molecule drugs, nucleic acids, proteins, peptides, polysaccharides, inorganic ions, radionuclides, and mixtures thereof.

In certain embodiments, the polymer conjugate is an acetylated carboxymethylcellulose covalently linked to at least one polyethylene glycol and at least one therapeutic agent. Suitable therapeutic agents include chemotherapeutic agents. Representative therapeutic agents include paclitaxel (PTX), docetaxel (DTX), cabazitaxel (CBZ), larotaxel (LTX), camptothecin (CMT), and doxorubicin (DOX).

In another aspect, the invention provides methods for making polymer nanoparticles containing therapeutic material.

In one embodiment, the method includes:

(a) introducing a first stream (e.g., one or more first streams) comprising a therapeutic material in a first solvent into a channel, wherein the channel has a first region adapted for flowing one or more streams introduced into the channel and a second region for mixing the contents of the one or more streams;

(b) introducing a second stream (e.g., one or more second streams) comprising a polymer in a second solvent into the channel;

(c) flowing the one or more first streams and the one or more second streams from the first region of the channel into the second region of the channel; and

(d) mixing of the contents of the one or more first streams and the one or more second streams flowing in the second region of the channel to provide a third stream comprising polymer nanoparticles containing the therapeutic material.

Suitable polymers include natural polymers, synthetic polymers, semi-synthetic polymers, derivatives thereof, combinations thereof, and copolymers thereof. Representative polymers include polyethylene glycols, polylactides, polyglycolides, poly(lactide-co-glycolide)s, polyacrylates, polymethacrylates, poly(ε-caprolactone)s, polyorthoesters, polyanhydrides, polylysines, polyethyleneimines, celluloses, chitins, alginates, carboxymethyl celluloses, acetylated carboxymethylcelluloses, chitosans, and gelatins, derivatives thereof, combinations thereof, and copolymers thereof.

Suitable therapeutic materials include small molecule drugs, nucleic acids, proteins, peptides, polysaccharides, inorganic ions, radionuclides, and mixtures thereof. Suitable therapeutic materials include chemotherapeutic agents. Representative chemotherapeutic agents include paclitaxel (PTX), docetaxel (DTX), cabazitaxel (CBZ), larotaxel (LTX), camptothecin (CMT), and doxorubicin (DOX).

In certain embodiments of the above methods, the first solvent is an aqueous buffer.

In certain embodiments of the above methods, the second solvent is a water-miscible solvent.

In certain embodiments of the above methods, mixing the contents of the one or more first streams and the one or more second streams comprises varying the concentration or relative mixing rates of the one or more first streams and the one or more second streams.

In certain embodiments of the above methods, mixing the contents of the first and second streams comprises chaotic advection. In certain embodiments of the above methods, the second region of the microchannel comprises bas-relief structures. In certain embodiments of the above methods, the second region of the microchannel has a principal flow direction and one or more surfaces having at least one groove or protrusion defined therein, the groove or protrusion having an orientation that forms an angle with the principal direction. In certain embodiments of the above methods, mixing the contents of the first and second streams comprises mixing with a micromixer.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a schematic illustration of a representative method of the invention for making nanoparticles of the invention. For example, for polymer nanoparticles containing a therapeutic material: polymer-solvent and therapeutic material-aqueous solutions are pumped into inlets of a microfluidic mixing device; herringbone features in the device induce chaotic advection of the stream and cause the polymer species to rapidly mix with the aqueous stream and form polymer nanoparticles. In representative devices, the mixing channel is 300 μm wide and 130 μm high, and the herringbone structures are 40 μm high and 53 μm thick.

FIGS. 2A and 2B show the mean particle diameter (2A) and polydispersity (PDI) (2B) plotted as a function of polymer conjugate concentration of a representative polymer conjugate nanoparticle (Cellax: acetylated carboxymethylcellulose polymer conjugated to polyethylene glycol and docetaxel, wherein the molar ratio of acetylated carboxymethycellulose acetyl groups: acetylated carboxymethycellulose carboxylic acid groups/polyethylene glycol/docetaxel is 2.18:0.82) manufactured using the methods and devices of the present invention. Cellax material was dissolved to the desired initial concentration in acetonitrile. Cellax nanoparticles were formed by mixing this solvent with an aqueous solution of 0.9% (w:w) sodium chloride in deionized water (0.9% NaCl). A total flow rate of 18 mL/min and a flow rate ratio of 3:1 (aqueous:solvent) was used. The resulting mixture was then immediately diluted 1:1 into 0.9% NaCl by pipetting to prevent particle aggregation, and dialyzed against 200 volumes of 0.9% NaCl to remove residual acetonitrile.

FIGS. 3A and 3B show the mean particle diameter (3A) and polydispersity (PDI) (3B) plotted as a function of representative manufacturing process condition for a representative polymer conjugate nanoparticle (Cellax: acetylated carboxymethylcellulose polymer conjugated to polyethylene glycol and docetaxel, wherein the molar ratio of acetylated carboxymethycellulose acetyl groups: acetylated carboxymethycellulose carboxylic acid groups/polyethylene glycol/docetaxel is 2.18:0.82) manufactured using the methods and devices of the present invention. All samples were manufactured at an initial Cellax concentration of 20 mg/mL in acetonitrile. An aqueous solution containing 0.9% NaCl was used; Process 7—total flow rate=18 mL/min, flow rate ratio (aqueous:organic)=5:1, no post-microfluidic dilution; Process 8—total flow rate=18 mL/min, flow rate ratio (aqueous:organic)=5:1, 1:1 post-microfluidic dilution with 0.9% NaCl; Process 9—total flow rate=18 mL/min, flow rate ratio (aqueous:organic)=3:1, no post-microfluidic dilution; Process 10—total flow rate=18 mL/min, flow rate ratio (aqueous:organic)=3:1, 1:1 post-microfluidic dilution with 0.9% NaCl.

### DETAILED DESCRIPTION OF THE INVENTION

The present invention provides methods and devices for manufacturing polymer particles. In certain embodiments, the polymer particles are nanoparticles that contain a therapeutic material. In other embodiments, the polymer particles are nanoparticles that contain a polymer conjugate, such as a polymer drug conjugate.

Methods for Making Polymer Nanoparticles Containing Therapeutic Material

In one embodiment, the invention provides a method for making polymer nanoparticles containing a therapeutic material, comprising:

(a) introducing a first stream (e.g., one or more first streams) comprising an therapeutic material in a first solvent into a fluidic device (e.g., a microfluidic device); wherein the device has a first region adapted for flowing one or more streams introduced into the device and a second region for mixing the contents of the one or more streams (e.g., with a microfluidic mixer);

(b) introducing a second stream (e.g., one or more second streams) comprising a polymer in a second solvent into the device;

(c) flowing the one or more first streams and the one or more second streams from the first region of the device into the second region of the device; and

(d) mixing of the contents of the one or more first streams and the one or more second streams in the second region of the device to provide a third stream comprising polymer nanoparticles containing the therapeutic material.

In another embodiment, the invention provides a method for making polymer nanoparticles containing a therapeutic material, comprising:

(a) introducing a first stream (e.g., one or more first streams) comprising a therapeutic material in a first solvent into a channel (e.g., microchannel); wherein the channel has a first region adapted for flowing one or more streams introduced into the channel and a second region for mixing the contents of the one or more streams;

(b) introducing a second stream (e.g., one or more second streams) comprising a polymer in a second solvent into the channel;

(c) flowing the one or more first streams and the one or more second streams from the first region of the channel into the second region of the channel; and

(d) mixing of the contents of the one or more first streams and the one or more second streams flowing in the second region of the channel to provide a third stream comprising polymer nanoparticles containing the therapeutic material.

In a further embodiment, the invention provides a method for making polymer nanoparticles containing a therapeutic material, comprising:

(a) introducing a first stream (e.g., one or more first streams) comprising a therapeutic material in a first solvent into a channel (e.g., microchannel); wherein the channel has a first region adapted for flowing one or more streams introduced into the channel and a second region for mixing the contents of the one or more streams;

(b) introducing a second stream (e.g., one or more second streams) comprising a polymer in a second solvent into the channel;

(c) flowing the one or more first streams and the one or more second streams from the first region of the channel into the second region of the channel while maintaining a physical separation of the two streams, wherein the one or more first streams and the one or more second streams do not mix until arriving at the second region of the channel; and

(d) mixing of the contents of the one or more first streams and the one or more second streams flowing in the second region of the channel to provide a third stream comprising polymer nanoparticles containing the therapeutic material.

Methods for Making Polymer Conjugate Nanoparticles

In a further embodiment, the invention provides a method for making polymer conjugate nanoparticles, comprising:

(a) introducing a first stream (e.g., one or more first streams) comprising a first solvent into a fluidic device (e.g., microfluidic) device; wherein the device has a first region adapted for flowing one or more streams introduced into the device and a second region for mixing the contents of the one or more streams (e.g., with a microfluidic mixer);

(b) introducing a second stream (e.g., one or more second streams) comprising a polymer conjugate in a second solvent into the device;

(c) flowing the one or more first streams and the one or more second streams from the first region of the device into the second region of the device; and

(d) mixing of the contents of the one or more first streams and the one or more second streams in the second region of the device to provide a third stream comprising polymer conjugate nanoparticles.

In another embodiment, the invention provides a method for making polymer conjugate nanoparticles, comprising:

(a) introducing a first stream (e.g., one or more first streams) comprising a first solvent into a channel (e.g., microchannel), wherein the channel has a first region adapted for flowing one or more streams introduced into the channel and a second region for mixing the contents of the one or more streams;

(b) introducing a second stream (e.g., one or more second streams) comprising a polymer conjugate in a second solvent into the channel;

(c) flowing the one or more first streams and the one or more second streams from the first region of the channel into the second region of the channel while maintaining a physical separation of the two streams, wherein the one or more first streams and the one or more second streams do not mix until arriving at the second region of the channel; and

(d) mixing of the contents of the one or more first streams and the one or more second streams flowing in the second region of the channel to provide a third stream comprising polymer conjugate nanoparticles.

In certain embodiments of the above methods, the fluidic device is a microfluidic device and the channels are microchannels.

In certain embodiments of the above methods, mixing the contents of the one or more first streams and the one or more second streams comprises varying the concentration or relative mixing rates of the one or more first streams and the one or more second streams.

In certain embodiments of the above methods, the methods further comprise diluting the third stream with an aqueous buffer. In certain embodiments, diluting the third stream comprises flowing the third stream and an aqueous buffer into a second mixing structure.

In certain embodiments of the above methods, the methods further comprise diafiltration of the aqueous buffer comprising polymer nanoparticles containing the therapeutic material with further aqueous buffer to reduce the amount of the second solvent. In certain embodiments of the above methods, the methods further comprise diafiltration of the aqueous buffer comprising polymer conjugate nanoparticles with further aqueous buffer to reduce the amount of the second solvent.

In the above methods, the first and second solvents may be the same or different. For example, in certain embodiments, the first and second solvents are different (e.g., the therapeutic material is in a first solvent that is an aqueous buffer and the polymer is in a second solvent that is a water-miscible solvent (e.g., an organic solvent)). In other embodiments, the first and second solvents are the same or substantially the same (e.g., both are aqueous solvents). In certain embodiments of the above methods, the first solvent is water or an aqueous buffer. Representative first solvents include citrate buffers, acetate buffers, phosphate buffers (phosphate buffered saline), and saline. Suitable second solvents include solvents in which the polymer conjugates are soluble and that are miscible with the first solvent. Suitable second solvents include water (e.g., aqueous buffers), organic acids, and alcohols. Representative second solvents include water, acetonitrile, methanol, ethanol, 1,4-dioxane, tetrahydrofuran, acetone, dimethylsulfoxide, and dimethylformamide.

In certain embodiments of the above methods, mixing the contents of the first and second streams comprises chaotic advection. In certain embodiments of the above methods, mixing the contents of the first and second streams comprises mixing with a micromixer.

In certain embodiments of the above methods, mixing of the one or more first streams and the one or more second streams is prevented in the first region by a barrier. In certain embodiments, the barrier is a channel wall, sheath fluid, or concentric tubing.

In certain embodiments of the above methods, the first and second streams flow under laminar flow conditions in the first region.

In a further embodiment, the invention provides a method for making polymer conjugate nanoparticles (e.g., polymer drug conjugate nanoparticles). The method includes

(a) introducing a first stream (e.g., one or more first streams) comprising a first solvent into a channel (e.g., microchannel); wherein the channel has a first region adapted for flowing one or more streams introduced into the channel and a second region for mixing the contents of the one or more streams;

(b) introducing a second stream (e.g., one or more second streams) comprising polymer conjugate in a second solvent into the channel to provide first and second streams flowing in the channel;

(c) flowing the one or more first streams and the one or more second streams from the first region of the channel into the second region of the channel; and

(d) mixing of the contents of the one or more first streams and the one or more second streams flowing in the second region of the channel to provide a third stream comprising polymer conjugate nanoparticles.

In one embodiment, the channel is a microchannel.

In certain embodiments, the channel is positioned in a fluidic device (e.g., a microfluidic device).

In one embodiment of the above method, the first and second streams flow under laminar flow conditions in the first region.

In the above method, the contents of the first and second streams can be mixed by chaotic advection. In one embodiment, mixing the contents of the one or more first streams and the one or more second streams comprises varying the concentration or relative mixing rates of the one or more first streams and the one or more second streams.

To further stabilize the third stream containing the polymer conjugate nanoparticles, the method can, but need not further include, comprising diluting the third stream with an aqueous buffer. In one embodiment, diluting the third stream includes flowing the third stream and an aqueous buffer into a second mixing structure. In another embodiment, the aqueous buffer comprising polymer conjugate nanoparticles is dialyzed to reduce the amount of the second solvent.

In this embodiment, suitable first solvents include water and aqueous buffers. Representative first solvents include citrate buffers, acetate buffers, phosphate buffers (phosphate buffered saline), and saline.

The second stream includes polymer conjugate in a second solvent. Suitable second solvents include solvents in which the polymer conjugates are soluble and that are miscible with the first solvent. Suitable second solvents include water (e.g., aqueous buffers), organic acids, and alcohols. Representative second solvents include water, acetonitrile, ethanol, 1,4-dioxane, tetrahydrofuran, acetone, dimethylsulfoxide, and dimethylformamide.

For the methods of the invention that are microfluidic mixing methods, these methods are distinguished from other microfluidic mixing methods in several ways. Whereas certain known methods require an equal or substantially equal proportion of aqueous and organic solvents (i.e., 1:1), the method of the invention generally utilizes a solvent ratio of aqueous to organic that exceeds 1:1. In certain embodiments, the solvent ratio of aqueous to organic is about 2:1. In certain embodiments, the solvent ratio of aqueous to organic is about 3:1. In certain embodiments, the solvent ratio of aqueous to organic is about 4:1. In certain other embodiments, the solvent ratio of aqueous to organic is about 5:1, about 10:1, about 50:1, about 100:1, or greater.

The polymer nanoparticles of the invention are advantageously formed in a microfluidic process that utilizes relatively rapid mixing and high flow rates. The rapid mixing provides polymer nanoparticles having the advantageous properties including size, homogeneity, and encapsulation efficiency. Mixing rates used in the practice of the method of the invention range from about 100 μsec to about 10 msec. Representative mixing rates include from about 1 msec to about 5 msec. Whereas hydrodynamic flow focusing methods operate at relatively low flow rates (e.g., 5 to 100 μL/minute) with relatively low polymer nanoparticle volumes, the methods of the invention operates at relatively high flow rates and relatively high polymer nanoparticle volumes. In certain embodiments, for methods that incorporate a single mixing region (i.e., mixer), the flow rate is about 1 to about 30 mL/min. For methods of the invention that utilize mixer arrays (e.g., 10 mixers), flow rates of 200 mL/minute are employed (for 100 mixers, flow rate 2000 mL/min). Thus, the methods of the invention can be readily scaled to provide quantities of polymer nanoparticles necessary for demanding production requirements. Coupled with the advantageous particle size, homogeneity, and encapsulation efficiencies realized, the method of the invention overcomes disadvantages of known microfluidic methods for producing polymer nanoparticles. One advantage of the methods of the invention for making the polymer nanoparticles is that the methods are scalable, which means that the methods do not change on scaling and that there is excellent correspondence on scaling.

Devices for Making Polymer Nanoparticles and Polymer Conjugate Nanoparticles

In another aspect, the invention provides devices (e.g., microfluidic devices) for producing polymer nanoparticles (e.g., polymer nanoparticles containing a therapeutic material, a polymer conjugate nanoparticles).

In one embodiment, the device (100) includes:

(a) a first inlet (102) for receiving a first solution (e.g., a first solvent comprising a therapeutic material);

(b) a first inlet channel (103) (e.g., microchannel) in fluid communication with the first inlet (so as to provide a first stream comprising the first solution);

(c) a second inlet (104) for receiving a second solution (e.g., a second solvent comprising a polymer);

(d) a second inlet channel (105) (e.g., microchannel) in fluid communication with the second inlet (so as to provide a second stream comprising the second solution);

(e) a third channel (110) (e.g., microchannel) for receiving the first and second streams, wherein the third channel has a first region (111) adapted for flowing the first and second streams introduced into the channel and a second region (112) adapted for mixing the contents of the first and second streams to provide a third stream (e.g., comprising polymer nanoparticles containing a therapeutic material). The third stream can be conducted from the device through outlet 120.

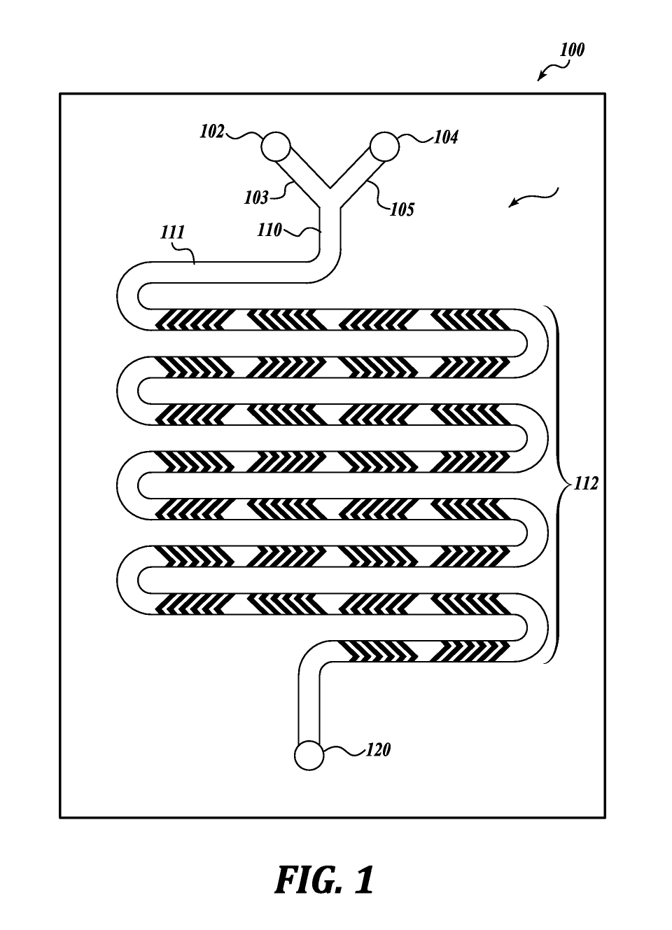

A representative device useful for carrying out the methods of the invention is illustrated schematically in FIG. 1. The reference numerals noted above refer to the device shown in FIG. 1.

In one embodiment, the device further includes means for diluting the third stream to provide a diluted stream comprising stabilized polymer conjugate nanoparticles containing a therapeutic material.

In one embodiment, the device further comprises means for diluting the third stream to provide a diluted stream (e.g., comprising stabilized polymer nanoparticles containing the therapeutic material). In certain embodiments, the means for diluting the third stream comprises a micromixer.

In certain embodiments, the device of the invention is a microfluidic device including one or more microchannels (i.e., a channel having its greatest dimension less than 1 millimeter). In certain embodiments, the microchannel has a diameter from about 20 to about 300 μm. As noted above, the channel has two regions: a first region for receiving and flowing at least two streams (e.g., one or more first streams and one or more second streams) into the device. The contents of the first and second streams are mixed in the microchannel's second region. In one embodiment, the second region of the channel (e.g., microchannel) comprises bas-relief structures. In one embodiment, the second region comprises a micromixer. In one embodiment, the second region of the microchannel has a principal flow direction and one or more surfaces having at least one groove or protrusion defined therein, the groove or protrusion having an orientation that forms an angle with the principal direction (e.g., a staggered herringbone mixer), as described in U.S. Application Publication No. 2004/0262223, expressly incorporated herein by reference in its entirety. In one embodiment, the second region of the microchannel comprises bas-relief structures. To achieve maximal mixing rates, it is advantageous to avoid undue fluidic resistance prior to the mixing region. Thus, one embodiment of the invention is a device in which non-microfluidic channels, having dimensions greater than 1000 microns, are used to deliver the fluids to a single mixing channel.

In one embodiment, the microfluidic device was produced by soft lithography, the replica molding of microfabricated masters in elastomer. The device has two inlets, one for each of the solutions prepared above, and one outlet. The device features a 300 μm wide and approximately 130 μm high mixing channel with herringbone structures formed by approximately 40 μm high and 75 μm thick features on the roof of the channel. The device was sealed using an oxygen plasma treatment to a 40×36×2 mm glass slide with three 1.5 mm holes drilled to match the inlet and outlet ports of the device.

In a second embodiment, microfluidic devices are produced from a hard thermoplastic such as cyclic olefin copolymer. A negative tool was machined using a CNC mill and devices formed using injection molding. Channel dimensions were preserved with the addition of a draft angle ranging between 1° and 5° on vertical surfaces.

Molded pieces were sealed to a blank substrate using a variety of techniques, including but not limited to, lamination, solvent welding, heat pressing and combinations thereof. Bonded devices were annealed to remove residual stresses from the production processes. Once formed, devices were installed and used in the custom instrument in the same way as elastomer devices.

In other embodiments, the first and second streams are mixed with other micromixers. Suitable micromixers include droplet mixers, T-mixers, zigzag mixers, multilaminate mixers, or other active mixers.

Mixing of the first and second streams can also be accomplished with means for varying the concentration and relative flow rates of the first and second streams.

In certain embodiments, the device further comprises means for varying the flow rates of the first and second streams.

In certain embodiments, the device further comprises a barrier effective to physically separate the one or more first streams from the one or more second streams in the first region.

To achieve maximal mixing rates it is advantageous to avoid undue fluidic resistance prior to the mixing region. Thus, in one embodiment, the device includes non-microfluidic channels (e.g., channels having dimensions greater than 1000 microns) that are used to deliver fluids to a single mixing channel. This device, which may be used for producing a polymer nanoparticle containing a therapeutic material or a polymer conjugate nanoparticle, includes:

(a) a single inlet microchannel for receiving both a first stream comprising a therapeutic material in a first solvent (or a first solvent) and a second stream comprising a polymer (or a polymer conjugate) in a second solvent;

(b) a second region adapted for mixing the contents of the first and second streams to provide a third stream comprising polymer nanoparticles containing a therapeutic material (or polymer conjugate nanoparticles).

In such an embodiment, the first and second streams are introduced into the microchannel by a single inlet or by one or two channels not having micro-dimensions, for example, a channel or channels having dimensions greater than 1000 μm (e.g., 1500 or 2000 μm or larger). These channels may be introduced to the inlet microchannel using adjacent or concentric macrosized channels.

Therapeutic Material

As used herein, the term “therapeutic material” is defined as a substance intended to furnish pharmacological activity or to otherwise have direct effect in the diagnosis, cure, mitigation, treatment or prevention of disease, or to have direct effect in restoring, correcting or modifying physiological functions.

Therapeutic materials include but are not limited to small molecule drugs, nucleic acids, proteins, peptides, polysaccharides, inorganic ions and radionuclides. A small molecule drug is a therapeutic agent having a molecular weight less than about 750 g/mole, less than about 500 g/mole, or less than about 350 g/mole.

Suitable therapeutic materials include therapeutic agents, such as chemotherapeutic agents (e.g., taxanes). Representative chemotherapeutic agents include paclitaxel (PTX), docetaxel (DTX), cabazitaxel (CBZ), larotaxel (LTX), camptothecin (CMT), and doxorubicin (DOX).

Polymers

As used herein, the term “polymer” refers to compounds comprising repeating units derived from polymerization of one or more monomers. A polymer prepared from a single monomer is a homopolymer. A polymer prepared from two or more monomers is a copolymer. A block copolymer is a copolymer that includes two or more blocks, where each block is a homopolymer or copolymer. Such polymers include any of numerous natural, synthetic and semi-synthetic polymers.

Natural polymers. The term “natural polymer” refers to any number of polymer species derived from nature. Such polymers include, but are not limited to, polysaccharides, such as cellulose, chitin, and alginate.

Synthetic polymers. The term “synthetic polymer” refers to any number of synthetic polymer species not found in nature. Such synthetic polymers include, but are not limited to, synthetic homopolymers and synthetic copolymers. Synthetic homopolymers include, but are not limited to, polyethylene glycols, polylactides, polyglycolides, polyacrylates, polymethacrylates, poly(ε-caprolactone)s, polyorthoesters, polyanhydrides, polylysine, and polyethyleneimines. “Synthetic copolymer” refers to any number of synthetic polymer species made up of two or more synthetic homopolymer subunits. Such synthetic copolymers include, but are not limited to, poly(lactide-co-glycolide)s, poly(lactide)-poly(ethylene glycol)s, poly(lactide-co-glycolide)-poly(ethylene glycol)s, and poly(ε-caprolactone)-poly(ethylene glycol)s.

Semi-synthetic polymers. The term “semi-synthetic polymer” refers to any number of polymers derived by the chemical or enzymatic treatment of natural polymers. Such polymers include, but are not limited to, carboxymethylcelluloses, acetylated carboxymethylcelluloses, cyclodextrins, chitosans, and gelatins. In one embodiment, the polymer is a carboxymethylcellulose. In another embodiment, the polymer is acetylated carboxymethylcellulose.

Polymer conjugate. As used herein, the term “polymer conjugate” refers to polymer to which one or more molecular species (e.g., therapeutic agent) are covalently, or non-covalently, coupled (i.e., molecular species is conjugated to the polymer). Such polymer conjugates include, but are not limited to, polymer drug conjugates (also referred to herein as polymer-therapeutic material conjugates). “Polymer-therapeutic material conjugate” or “polymer drug conjugate” refers to a polymer conjugate in which one or more of the conjugated molecular species is a therapeutic material or drug. Representative drugs include therapeutic agents such as chemotherapeutic agents (e.g., paclitaxel (PTX), docetaxel (DTX), cabazitaxel (CBZ), larotaxel (LTX), camptothecin (CMT), and doxorubicin (DOX)). Suitable polymer drug conjugates include, but are not limited to, cellulose-based drug conjugates. A representative cellulose-based drug conjugate is an acetylated carboxymethylcellulose (CMC-Ac) covalently linked to at least one poly(ethylene glycol) (PEG) and at least one drug (e.g., hydrophobic drug). Suitable cellulose-based drug conjugates are described in U.S. Pat. No. 8,591,877 and WO 2014/015422, each expressly incorporated herein by reference in its entirety. These cellulose-based drug conjugates are referred to as “Cellax” conjugates. Representative acetylated carboxymethylcellulose-polyethylene glycol-drug conjugates useful in the methods of the invention include Cellax-PTX, Cellax-DTX, Cellax-CBZ, Cellax-LTX, Cellax-CMT, and Cellax-DOX.

The following is a description of a representative method and device for making polymer conjugate nanoparticle systems.

Rapid mixing provides production of monodisperse polymer conjugate nanoparticles. Formulation of polymer conjugate nanoparticles was performed by rapidly mixing a polymer conjugate-acetonitrile solution with an aqueous buffer inside a microfluidic mixer (FIG. 1) designed to induce chaotic advection and provide a controlled mixing environment. The fluidic channel contains herringbones that generate a chaotic flow by changing the orientation of herringbone structures between half cycles, causing a periodic change in the centers of local rotational and extensional flow.

The following representative example utilizes the polymer conjugate comprising an acetylated carboxymethylcellulose polymer conjugated to polyethylene glycol and docetaxel, wherein the molar ratio of acetylated carboxymethycellulose acetyl groups: acetylated carboxymethycellulose carboxylic acid groups/polyethylene glycol/docetaxel is 2.18:0.82. This polymer conjugate is referred to in this example as “Cellax.”

Cellax was solubilized in acetonitrile and mixed with an aqueous buffer containing 0.9% w:w sodium chloride in deionized water (0.9% NaCl) using the microfluidic mixing device. The formed Cellax nanoparticles were diluted 1:1 into 0.9% NaCl immediately following formation to reduce ethanol content to approximately 12.5 vol %.

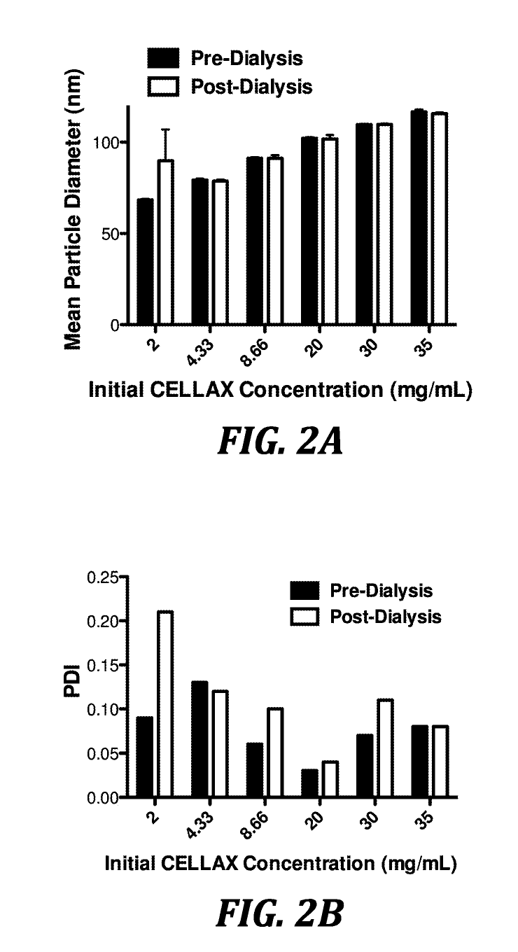

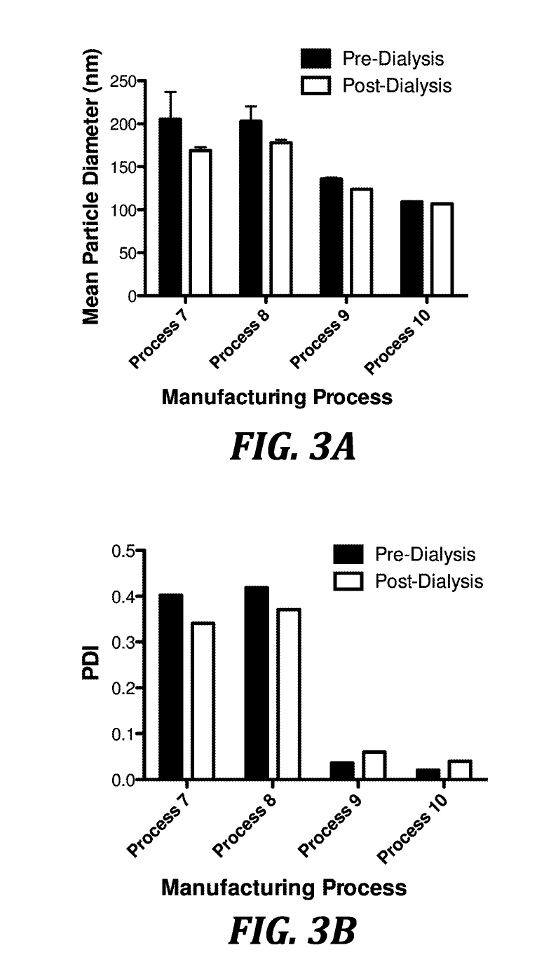

The results shown in FIGS. 2A and 2B and FIGS. 3A and 3B demonstrate that a microfluidic device containing a staggered herringbone mixer can be used to generate monodispersed nanoparticles using polymer drug conjugates. The resulting nanoparticles are extremely sensitive to process conditions including polymer concentration, total flow rate, flow rate ratio, and post-microfluidic dilution.

The fluidic devices and methods of the invention allow for use of Cellax to form Cellax nanoparticles of 100 nm size or smaller. With regard to formation of Cellax nanoparticles, the rate and ratio of mixing are important parameters. Rapid mixing of the acetonitrile-polymer conjugate solution with aqueous buffer results in an increased polarity of the medium that reduces the solubility of dissolved polymer conjugate, causing them to precipitate out of solution and form nanoparticles. Rapid mixing causes the solution to quickly achieve a state of high supersaturation of polymer conjugates throughout the entire mixing volume, resulting in the rapid and homogeneous nucleation of nanoparticles. Increased nucleation and growth of nanoparticles depletes the surrounding liquid of free polymer conjugate, thereby limiting subsequent growth by the aggregation of free polymer.

The polymer nanoparticles and methods for making the nanoparticles of the invention described herein include (i.e., comprise) the components and steps recited. In certain embodiments, the polymer nanoparticles and methods of the invention include the recited components and other additional components that do not affect the characteristics of the particles and methods (i.e., the polymer nanoparticles and methods consist essentially of the recited components). Additional components that affect the polymer nanoparticle and method characteristics include components such as additional materials or steps that disadvantageously alter or affect therapeutic profile and efficacy of the particles, additional components or steps that disadvantageously alter or affect the ability of the particles to solubilize the recited therapeutic components, and additional components or steps that disadvantageously alter or affect the ability of the particles to increase the cellular uptake or bioavailability of the recited therapeutic components. In other embodiments, the polymer nanoparticles and methods of the invention include only (i.e., consist of) the recited components or steps.

The following examples are provided for the purpose of illustrating, not limiting, the claimed invention.

### EXAMPLES

### Example 1

### Preparation of Polymer Conjugate Nanoparticles: Vortex Mixing Method

In the example, the preparation of polymer conjugate nanoparticles using the vortex (bulk) mixing method is described.

Cellax polymer conjugate nanoparticles were prepared according to the method described in Ernsting et al., “A docetaxel-carboxymethylcellulose nanoparticle outperforms the approved taxane nanoformulation, Abraxane, in mouse tumor models with significant control of metastases,” Journal of Controlled Release, Volume 162, Issue 3, 28 Sep. 2012, Pages 575-581. 10 mg of Cellax polymer conjugate (acetylated carboxymethylcellulose polymer conjugated to polyethylene glycol and docetaxel, at a molar ratio of acetylated carboxymethycellulose acetyl groups: acetylated carboxymethycellulose carboxylic acid groups/polyethylene glycol/docetaxel is 2.18:0.82) was dissolved in acetonitrile (MeCN, 1 mL), at a final polymer conjugate concentration of 10 mg/mL, and pipetted into vortexing 0.9% saline. The resulting particle solution was dialyzed against 0.9% saline overnight in a Slide-A-Lyzer 10,000 MWCO cartridge, filtered through a 0.22-μm Millipore PVDF filter, and concentrated using a Vivaspin 20 unit (MWCO 10,000). Particle size and zeta potential were measured with a Malvern Zetasizer (Nano-ZS, Malvern Instruments, Malvern, UK).

### Example 2

### Preparation of Polymer Conjugate Nanoparticles: Microfluidic Mixer Method

In the example, a representative polymer conjugate is used to prepare polymer conjugate nanoparticles using a method of the invention is described.

Solutions were prepared at a concentration of 0.5 mg/mL to 8.75 mg/mL polymer conjugate. A polymer conjugate solution containing acetylated carboxymethylcellulose polymer conjugated to polyethylene glycol and docetaxel, at a molar ratio of acetylated carboxymethycellulose acetyl groups: acetylated carboxymethycellulose carboxylic acid groups/polyethylene glycol/docetaxel of 2.18:0.82 was dissolved in acetonitrile at concentrations from 2-35 mg/mL. FIG. 1 is a schematic illustration of the microfluidic apparatus used in this example. The device has two inlets, one for the solutions prepared above, and one for aqueous buffer, and one outlet. The microfluidic device was produced by soft lithography, the replica molding of microfabricated masters in elastomer. The device features a 300 μm wide and approximately 130 μm high mixing channel with herringbone structures formed by approximately 40 μm high and 75 μm thick features on the roof of the channel. The device was sealed using an oxygen plasma treatment to a 40×36×2 mm glass slide with three 1.5 mm holes drilled to match the inlet and outlet ports of the device. The bonded device was installed into a custom instrument, having a top plate with o-rings to seal the device to the instrument, and a back plate with luer fitting for loading reagents in syringes. Once the device and reagents were loaded, the instrument acted as a syringe pump to dispense the fluid at the prescribed rate through the device. The flow rate of each stream was varied from 3 ml/min to 15 ml/min. The instrument introduces the two solutions into the microfluidic device where they come into contact at a Y-junction. Insignificant mixing occurs under laminar flow by diffusion at this point, whereas the two solutions become mixed as they pass along the herringbone structures and around the serpentine channels.

Mixing occurs in these structures by chaotic advection, causing the characteristic separation of laminate streams to become increasingly small, thereby promoting rapid diffusion. This mixing occurs on a millisecond time scale and results in the polymer conjugates being transferred to a progressively more aqueous environment, reducing their solubility and resulting in the spontaneous formation of nanoparticles. Polymer nanoparticles were formed at total flow rates from 12-18 mL/min and aqueous: solvent flow ratios of 3:1-5:1. Following mixing in the microfluidic device, the polymer conjugate nanoparticle was generally diluted into a polystyrene vial containing one volume of buffer by rapid pipetting. Residual acetonoitrile is finally removed through dialysis to 0.9% NaCl.

Particle size and polydispersity was determined by dynamic light scattering using a Malvern Malvern Zetasizer (Nano-ZS, Malvern Instruments, Malvern, UK). Number-weighted and intensity-weighted distribution data was used.

While illustrative embodiments have been illustrated and described, it will be appreciated that various changes can be made therein without departing from the spirit and scope of the invention.

1. A method for making polymer conjugate nanoparticles, comprising:
(a) introducing a first stream comprising a first solvent into a channel, wherein the channel has a first region adapted for flowing one or more streams introduced into the channel and a second region for mixing the contents of the one or more streams;
(b) introducing a second stream comprising polymer conjugate in a second solvent into the channel to provide first and second streams flowing in the channel;
(c) flowing the one or more first streams and the one or more second streams from the first region of the channel into the second region of the channel; and
(d) mixing of the contents of the one or more first streams and the one or more second streams flowing in the second region of the channel to provide a third stream comprising polymer conjugate nanoparticles.

2. The method of claim 1, wherein mixing the contents of the one or more first streams and the one or more second streams comprises varying the concentration or relative mixing rates of the one or more first streams and the one or more second streams.

3. The method of claim 1 or 2 further comprising diluting the third stream with an aqueous buffer.

4. The method of claim 3, wherein diluting the third stream comprises flowing the third stream and an aqueous buffer into a second mixing structure.

5. The method of any one of claims 1-4 further comprising diafiltration of the third stream to reduce the amount of the second solvent.

6. The method of any one of claims 1-5, wherein the first solvent is an aqueous buffer.

7. The method of any one of claims 1-6, wherein the second solvent is a water-miscible solvent.

8. The method of any one of claims 1-7, wherein mixing the contents of the first and second streams comprises chaotic advection.

9. The method of any one of claims 1-8 wherein the second region of the microchannel comprises bas-relief structures.

10. The method of any one of claims 1-8, wherein the second region of the microchannel has a principal flow direction and one or more surfaces having at least one groove or protrusion defined therein, the groove or protrusion having an orientation that forms an angle with the principal direction.

11. The method of any one of claims 1-8, wherein mixing the contents of the first and second streams comprises mixing with a micromixer.

12. The method of claim 1, where the polymer conjugate comprises a polymer selected from natural polymers, synthetic polymers, semi-synthetic polymers, derivatives thereof, combinations thereof, and copolymers thereof.

13. The method of claim 1, where the polymer conjugate comprises a polymer selected from a polyethylene glycol, polylactide, polyglycolide, poly(lactide-co-glycolide), polyacrylate, polymethacrylate, poly(ε-caprolactone), polyorthoester, polyanhydride, polylysine, polyethyleneimine, cellulose, chitin, alginate, carboxymethyl cellulose, acetylated carboxymethylcellulose, chitosan and gelatin, derivatives thereof, combinations thereof, and copolymers thereof.

14. The method of claim 1, wherein the polymer conjugate comprises a therapeutic material selected from one or more small molecule drugs, nucleic acids, proteins, peptides, polysaccharides, inorganic ions, radionuclides, and mixtures thereof.

15. The method of claim 1, wherein the polymer conjugate is an acetylated carboxymethylcellulose covalently linked to at least one polyethylene glycol and at least one therapeutic agent.

16. The method of claim 15, wherein the therapeutic agent is a chemotherapeutic agent.

17. The method of claim 15, wherein the therapeutic agent is selected from paclitaxel (PTX), docetaxel (DTX), cabazitaxel (CBZ), larotaxel (LTX), camptothecin (CMT), and doxorubicin (DOX).

18. A method for making polymer particles containing a therapeutic material comprising:
(a) introducing a first stream comprising a therapeutic material in a first solvent into a channel, wherein the channel has a first region adapted for flowing one or more streams introduced into the channel and a second region for mixing the contents of the one or more streams;
(b) introducing a second stream comprising a polymer in a second solvent into the channel;
(c) flowing the one or more first streams and the one or more second streams from the first region of the channel into the second region of the channel; and
(d) mixing of the contents of the one or more first streams and the one or more second streams flowing in the second region of the channel to provide a third stream comprising polymer nanoparticles containing the therapeutic material.

19. The method of claim 18, wherein mixing the contents of the one or more first streams and the one or more second streams comprises varying the concentration or relative mixing rates of the one or more first streams and the one or more second streams.

20. The method of claim 18 or 19 further comprising diluting the third stream with an aqueous buffer.

21. The method of claim 20, wherein diluting the third stream comprises flowing the third stream and an aqueous buffer into a second mixing structure.

22. The method of any one of claims 18-21 further comprising diafiltration of the third stream to reduce the amount of the second solvent.

23. The method of any one of claims 18-22, wherein the first solvent is an aqueous buffer.

24. The method of any one of claims 18-23, wherein the second solvent is a water-miscible solvent.

25. The method of any one of claims 18-24, wherein mixing the contents of the first and second streams comprises chaotic advection.

26. The method of any one of claims 18-25 wherein the second region of the microchannel comprises bas-relief structures.

27. The method of any one of claims 18-25, wherein the second region of the microchannel has a principal flow direction and one or more surfaces having at least one groove or protrusion defined therein, the groove or protrusion having an orientation that forms an angle with the principal direction.

28. The method of any one of claims 18-25, wherein mixing the contents of the first and second streams comprises mixing with a micromixer.

29. The method of claim 18, where the polymer is selected from natural polymers, synthetic polymers, semi-synthetic polymers, derivatives thereof, combinations thereof, and copolymers thereof.

30. The method of claim 18, where the polymer is selected from a polyethylene glycol, polylactide, polyglycolide, poly(lactide-co-glycolide), polyacrylate, polymethacrylate, poly(ε-caprolactone), polyorthoester, polyanhydride, polylysine, polyethyleneimine, cellulose, chitin, alginate, carboxymethyl cellulose, acetylated carboxymethylcellulose, chitosan and gelatin, derivatives thereof, combinations thereof, and copolymers thereof.

31. The method of claim 18, wherein the therapeutic material is selected from one or more small molecule drugs, nucleic acids, proteins, peptides, polysaccharides, inorganic ions, radionuclides, and mixtures thereof.

32. The method of claim 18, wherein the therapeutic material is a chemotherapeutic agent.

33. The method of claim 18, wherein the therapeutic material is selected from paclitaxel (PTX), docetaxel (DTX), cabazitaxel (CBZ), larotaxel (LTX), camptothecin (CMT), and doxorubicin (DOX).
